(19) United States
(12) Patent Application Publication
(10) Pub. No.: US 2016/0083757 A1
(43) Pub. Date: Mar. 24, 2016

(54) ENZYMATIC PROCESS FOR THE PRODUCTION OF MANNOSYLERYTHRITOL LIPIDS FROM LIGNOCELLULOSIC MATERIALS

(71) Applicants: INSTITUTO SUPERIOR TECNICO, Lisboa (PT); LABORATÓRIO NACIONAL DE ENERGIA E GEOLOGIA, Alfragide (PT)

(72) Inventors: Cesar Simoes da FONSECA, Alges (PT); Nuno Ricardo FARIA, Alges (PT); Frederico Castelo Alves FERREIRA, Lisboa (PT)

(21) Appl. No.: 14/891,859

(22) PCT Filed: May 15, 2014
(86) PCT No.: PCT/PT2014/000032

§ 371 Date: Nov. 17, 2015

(30) Foreign Application Priority Data
May 17, 2013 (PT) 106959

(51) Int. Cl. C12P 7/64

(52) U.S. Cl. CPC C12P 7/64; C12P 2203/00; C12P 2201/00

(57) ABSTRACT

The present invention relates to processes for the production of microbial glycolipids, mannosylerythritol lipids (MEL), from lignocellulosic carbon source. These processes are characterized in that the use of lignocellulosic materials for the production of a microbial glycolipids, MEL, comprising a fermentation preferably using fungi of the genus Pseudozyma or other microorganisms such as genetically modified fungi or bacteria. The processes for production of microbial glycolipids, MEL comprise three steps: pretreatment of lignocellulosic material; enzymatic hydrolysis; and fermentation. The enzymatic hydrolysis and fermentation may take place sequentially or simultaneously with addition of exogenous enzymes or simultaneously with enzymes produced by the microorganism itself. The produced microbial glycolipids have applications as: biosurfactants; antimicrobials; anticancer agents; wound healing factors; stabilizer agents on storage and purification of proteins or vaccines; drugs and gene deliver agents; antifreeze agents.

### FIELD OF THE INVENTION

### Technical Field to Which the Invention Relates

The present invention relates to processes for the production of microbial glycolipids, mannosylerythritol lipids (MEL), from lignocellulosic carbon sources comprising cellulose and hemicellulose.

The biological synthesis of fatty acids typically results in lipids with chains of 16 and 18 carbons length, being palmitic acid and stearic acid the saturated fatty acids most abundant in nature, where they are used as energy reserves and precursors of cellular components, such as phospholipids and glycolipids. The microbial glycolipids have unique properties because they comprise a hydrophilic glycosidic component and a hydrophobic lipidic component. These characteristics provide the glycolipid biosurfactants with properties that are dependent on, among other factors, the length of the lipidic chain(s). The length(s) of the lipidic component of microbial glycolipids is variable and depends on the glycolipid and on the microorganism responsible for their synthesis.

Thus: the sophorolipids and cellobiolipids comprise lipidic chains of 16 and 18 carbons length; The rhamnolipids and mannosylerythritol lipids comprise shorter lipidic chains of 6 to 16 carbons length; and trealolipids lipid comprise lipidic chains of variable size, typically greater than 16 carbons length, reaching up to 50 carbons length (1). The microbial production of glycolipids is described as using oils and alkanes as carbon source and, in some cases, glycerol or glucose (1). The use of oils as substrate has drawbacks concerning the technological process for glycolipids production, in particular regarding the steps required for separation of the product from the fermentation broth, more specifically the isolation of the glycolipids from the residual oils (1).

Conversely, the substrates typically used for the production of glycolipids do not contribute for process and product sustainability, as they have high commercial value (e.g. glucose), compete directly with the food value chain and/or are obtained from dedicated crops with high environmental impact associated to land use for cultivation (e.g. soybean oil).

The present invention relates to processes for the production of microbial glycolipids, MEL, from lignocellulosic carbon sources. These carbon sources, obtained from agricultural, forestry and/or agro-industrial wastes, have low commercial value and represent one of the most sustainable options for the production of added value products, such is the case of microbial glycolipids.

The processes mentioned for the production of microbial glycolipids comprise three steps: pretreatment of lignocellulosic material; enzymatic hydrolysis; and fermentation. The enzymatic hydrolysis and fermentation may take place sequentially or simultaneously with the addition of exogenous enzymes or simultaneously with enzymes produced by the microorganism itself. Preferred microorganisms for the production of these glycolipids are yeasts of Pseudozyma genus.

The glycolipids produced under these processes may have applications in medicine and in the pharmaceutical, chemical, cosmetic, biotechnology, food and nutraceutical industries. These glycolipids have antimicrobial activity particularly against gram positive bacteria, the apoptosis inducing activity and/or differentiation of animal cells, regeneration of cell viability in epithelial cell models, high affinity for glycoproteins with potential application for human immunoglobulin purification, ability to form thermodynamically stable vesicles with potential application for drug and gene delivery and antifreeze properties (1).

### State of the Art

The mannosylerythritol lipids (MEL) are glycolipids comprised of a mannosylerythritol glycosidic component, which can be deacetylated, mono- or di-acetylated and by a lipidic component comprised of two chains containing 6 to 14 carbons linked to the glycosidic component (2).

These glycolipids are produced by fungi of the Pseudozyma and Ustilago genera (3). They have wide applications as biosurfactant and can be used in medicine and pharmaceutical, chemical, cosmetic, biotechnological, food and nutraceutical industries (2,4).

The most commonly used substrate for MEL production by strains of the Pseudozyma genus is soybean oil. Other substrates described as suitable for MEL production are sunflower oil, glycerol and alkanes (5,6,7). The international application WO2004/0020647 discloses a process for the production of glycolipids using soybean oil as substrate (8). However, the use of soybean oil and other oils, presents additional drawbacks with concerning the technological process, as well as process and product sustainability. The presence of oil in the fermentation broth, as well as their degradation products, hinder the process for recovery and purification of the glycolipid produced, requiring several additional steps of extraction with organic solvents for separation of the glycolipid, which makes the process more complex and leads to a decrease in glycolipid recovery yield. Moreover, vegetable oils, such as soybean oil, are also used in the food industry, implying direct competition for this raw material with the food supply chain. Additionally, the use of dedicated crops for the production of oil makes these substrates unsustainable according to the current sustainability criteria, which accounts for greenhouse gases emissions related to land change use for dedicated crops.

The use of water soluble substrates is an alternative for the production of glycolipids, bringing advantages in the recovery processes. Accordingly, the use of glucose as carbon source for the production of mannosylerythritol lipids has been reported (9), with advantages in the recovery processes of the glycolipid from the fermentation broth.

The lignocellulosic material, in particular cellulose and hemicellulose, which is converted during the pretreatment and/or enzymatic hydrolysis into mono-, di- or oligosaccharides, such as cellodextrins, xylo- oligosaccharide, cellobiose, xylobiose, D-glucose, D-mannose, D-galactose, D-xylose and L-arabinose, is an inexpensive and renewable substrate and has low-energy harvesting costs and is composed of fermentable sugars. According to nature and composition of the lignocellulosic material, different physical and/or chemical pretreatment processes may be applied, with or without the use of acids, bases, organic solvents and/or water, combining different temperatures and reaction times, usually between 100 and 250° C. for periods between 10 and 30 minutes.

According to the type of material and pretreatment, the fractions rich in sugars, such as cellulose and hemicellulose, are released and hydrolyzed to a greater or lesser extent (10). For some lignocellulosic materials the pretreatment step may not be required. The cellulose and hemicellulose and/or their respective hydrolysates may be subjected to enzymatic hydrolysis resulting in fermentable sugars, for example mono-, di - or oligosaccharides, celodextrins, xylooligosaccharides, cellobiose, xylobiose, D-glucose, D-mannose, D-galactose, D-xylose and L-arabinose among others (10).

The lignocellulosic residues, including the ones from agricultural, forestry or agro-industrial origin, are, in fact, a sustainable option with growing economic and environmental interest for the production of biofuels, biopolymers and other bioproducts, within a concept of biorefinery where the production of biosurfactants, in particular glycolipids, can be included (11, 12, 13).

### SUMMARY OF THE INVENTION

The present invention relates to processes for the production of microbial glycolipids, mannosylerythritol lipids (MEL), from carbon sources of lignocellulosic origin including cellulose, hemicellulose or mono -, di- or oligosaccharides resulting from their hydrolysis, for example cellodextrins, xylo- oligosaccharide, cellobiose, xylobiose, D-glucose, D-mannose , D-galactose, D-xylose and L-arabinose.

The process for glycolipids production comprises three steps: pretreatment of lignocellulosic material; enzymatic hydrolysis; and fermentation. The enzymatic hydrolysis and fermentation process may take place: (i) sequentially, in different reaction vessels or in the same reaction vessel, in both cases with the addition of exogenous enzymes; (ii) simultaneously in the same reaction vessel without addition of exogenous enzymes, that is by action of endogenous enzymes produced by the microorganism used in the fermentation process or (iii) simultaneously in the same reaction vessel, with the addition of exogenous enzymes.

The aim of the present invention is the use of renewable carbon sources of low commercial value, such as lignocellulosic materials, for the sustainable production of microbial glycolipids MEL.

### DETAILED DESCRIPTION OF THE INVENTION

The present invention relates to processes for the production of microbial glycolipid, mannosylerythritol lipids (MEL), from renewable carbon sources of low commercial value, such as those of lignocellulosic origin, comprised of cellulose and hemicellulose.

The MEL are characterized in that having a glycosidic mannosylerythritol component, with different variants of acetylation, where two lipidic chains, typically composed of 6 to 14 carbons, are linked.

The MEL production processes are characterized in that the use of lignocellulosic materials that include, but are not limited to wheat straw, sugar cane straw, corn stover, corn cobs, rice straw, rice husk, sorghum straw, sweet sorghum straw, barley straw, oat straw, rye straw, triticale straw, cottonseed hulls, coffee husk, bamboo, pine wood, pine bark, other wood of conifers (cypress, cedar, araucaria, fir, spruce), eucalyptus wood, eucalyptus bark, other wood of angiosperms (ash, beech, birch, poplar, oak, willow, maple, olive tree), herbaceous biomass (hay, grass, seaweed), sugar cane bagasse, olive pomace, brewery spent grain, wastes from wood processing industry (wood chips, sawdust), waste paper (newspaper, office paper), recycled paper sludge, other residues from pulp and paper industry (primary sludge, sulfite liquors) and municipal solid wastes.

The glycolipids production processes include three steps. The first step comprises the pretreatment of lignocellulosic material, in order to increase the accessibility of hydrolytic enzymes to the cellulose and/or hemicellulose in the second step; the use of some lignocellulosic materials may not require the first step. The second step concerns enzymatic hydrolysis of cellulose and hemicellulose components by cellulolytic and/or hemicellulolytic enzymes. The third step concerns the fermentation, preferably using fungi of the Pseudozyma genus. Regarding the second and third steps, three process configurations are considered for the production of glycolipids, MEL, respectively: (i) separated hydrolysis and fermentation (SHF), where the second and third steps occur sequentially in different reaction vessels or in the same vessel, and conditions for each step are optimized for each individual step alone and then for the interaction between them; (ii) simultaneous saccharification and fermentation (SSF), in which the second and third steps occur simultaneously in the same reaction vessel, with the inherent advantages of process intensification, such as reducing product inhibition of enzymatic hydrolysis due to its simultaneously consumption in fermentation, and reduction of process time when compared to SHF. However, the conditions selected may impair operation at optimal conditions of each individual step; (iii) a consolidated bioprocessing (CBP), in which the second and third steps occur simultaneously in the same reaction vessel, without addition of exogenous enzymes, taking advantage of the enzymes produced by the microorganism itself.

The current invention covers the use of different physical and/or chemical pretreatment processes, with or without the presence of acids, bases, organic solvents and/or water, and the combination of different temperatures and reaction times, usually between 100 and 250° C. and residence times, which, depending on the process, varies between 0 and 300 minutes. The lignocellulosic material, in particular cellulose and hemicellulose polysaccharides, which, after pretreatment with acids, bases, organic solvents and/or water and/or enzymatic hydrolysis are converted to mono-, di- or oligosaccharides such as cellodextrins, xylo-oligosaccharides, cellobiose, xylobiose, D-glucose, D-mannose, D-galactose, D-xylose and L-arabinose, are then used as carbon source(s) for the fermentation step.

When exogenous enzymes or their mixtures are added into the process, it may be included a purification step, for example a membrane process, such as dialysis, to remove compounds with inhibitory effects on cell activity, cell proliferation and/or bioconversion mediated by the fungi used. The fermentation process of the current invention can use microorganisms, in this case, different kinds of fungi, which include, but are not limited to, Pseudozyma, Ustilago, Sporisorium, Moesziomyces, Macalpinomyces, preferably of the Pseudozyma genus, and most preferably of the species Pseudozyma antarctica and Pseudozyma aphidis; or its genetically modified variants, or other microorganisms such as fungi or bacteria genetically modified which include, but are not limited to the genera Saccharomyces, Pichia, Pseudozyma, Ustilago, Escherichia and Bacillus.

The microbial glycolipids MEL are produced by fungi, preferably of the genus Pseudozyma, and most preferably of the species Pseudozyma antarctica and Pseudozyma aphidis from lignocellulosic material under aerobic conditions at temperatures between 4-40° C., preferably at 27° C., using a nitrogen source, preferably nitrate, in batch or fed-batch mode.

The enzymatic hydrolysis and fermentation process may take place sequentially, in different reaction vessels or in the same vessel, or simultaneously in the same reaction vessel with or without addition of exogenous enzymes, which include but are not limited to cellulases, cellobiohydrolases, glucosidases, xylanases, xylosidases and arabinofuranosidase. The addition of exogenous enzymes can be minimized due to the ability of the microorganisms to produce their own hydrolytic enzymes. Considering the industrial production and application of MEL, this process has the following advantages compared to other existing processes:

1—Production of glycolipids, MEL from sustainable and low-cost carbon sources such as lignocellulosic materials: wheat straw, sugar cane bagasse, sugar cane straw, corn stover, rice straw, rice husk, brewery spent grain and paper sludge.

2—In addition to glucose derived from cellulose, this process allows for the conversion of an additional fraction of the lignocellulosic biomass, the xylose resulting from hemicellulose hydrolysis, into glycolipids, with similar yields to those described for glucose.

3—Microorganisms capable producing glycolipids of MEL can also be used as producers of their own hydrolytic enzymes, including but not limited to xylanases and xylosidases, reducing the cost associated with addition of exogenous enzymes used for the enzymatic hydrolysis step.

4—In comparison with the processes for glycolipids production from oils, in particular soybean oil, the production of glycolipids, MEL, from sugars allows for a more efficient separation of the glycolipid fraction from the culture medium.

5—Glycolipids, MEL, contrary to the intracellular lipids or the structural lipids of cell membrane, are excreted, which provides advantages concerning increased yield and facilitated isolation from fermentation broth.

### EXAMPLES

### 1. Glycolipid Production From Xylose

### 1.A. Pre-Culture for Cell Growth

Medium:
Glucose, 40 g/L;
NaNO₃, 3 g/L;
KH₂PO₄, 0.3 g/L;
MgSO₄.7H₂O, 0.3 g/L;
Yeast extract, 1 g/L;

All compounds were prepared in concentrated solutions and autoclaved at 121° C. for 20 minutes. After cooling, the compounds solutions mentioned were diluted under sterile conditions with sterile distilled water in an Erlenmeyer flask sterile solution to reach the concentrations described above. The medium was inoculated with Pseudozyma antarctica PYCC 5084T biomass supplied by the Portuguese Yeast Culture Collection (PYCC), CREM, FCT/UNL, and inoculated for 2 days under aerobic conditions with constant mixing and fermentation temperature of 27° C.

### 1.B. Fermentation Process for Glycolipid Production

Medium:
Xylose, 40 g/L;
KH₂PO₄, 0.3 g/L;
MgSO₄.7H₂O, 0.3 g/L;
Yeast extract, 1 g/L;

The culture medium for fermentation was prepared with sterile water at an initial pH of 6. The culture medium was then inoculated with 10% (v/v) of pre-culture prepared as described in 1.A., and incubated under aerobic conditions with constant mixing at 27° C. for 14 days. The biomass was quantified by dry weight measurements. The culture reached its maximum biomass (approx. 10 g/L) at 48 hours, remaining constant from that point forward. MEL production was measured after 4 days and reached the maximum at day 14, at a value of 4.5 g/L (mean value of 3 experiments), and a yield of 0.11 (gMEL/gsubstrate) representing approximately 30% of the theoretical maximum expected value.

### 2. Production of Glycolipids From Mixtures of Glucose and Xylose

### 2.A. Pre-Culture

The pre-culture was prepared as described in 1.A.

### 2.B. Fermentation Process for Glycolipid Production

Medium:
Xylose 20 g/L;
Glucose, 20 g/L;
KH₂PO₄, 0.3 g/L;
MgSO₄.7H₂O, 0.3 g/L;
Yeast extract, 1 g/L;

The culture medium for fermentation was prepared with sterile water at an initial pH of 6. A mixture of xylose and glucose was here used as substrate, these sugars representing the most abundant monosaccharides constituting most of the lignocellulosic materials. The culture medium was inoculated with 10% (v/v) of pre-culture prepared as described in 2.A. and incubated under aerobic conditions, with constant mixing at 27° C. for 14 days. MEL production was measured after 4 days and reached the maximum at day 14 at a value of 4.6 g/L in MEL, and a yield of 0.12 (gMEL/gsubstrate), representing approximately 30% of the maximum value theoretical expected.

### 3. Glycolipid Production From Glucose With the Addition Substrate at Day 4 of Fermentation

### 3.A. Pre-Culture

The pre-culture was prepared as described in 1.A.

### 3.B. Fermentation Process for Glycolipid Production

Medium:
Glucose, 40 g/L;
KH₂PO₄, 0.3 g/L;
MgSO₄.7H₂O, 0.3 g/L;
Yeast extract, 1 g/L;
NaNO₃, 3 g/L;

The culture medium for fermentation was prepared with sterile water at an initial pH of 6. The culture medium was then inoculated with 10% (v/v) of pre-culture prepared in 3.A., and incubated under aerobic conditions with constant mixing at 27° C. for 14 days. The biomass was quantified by dry weight measurements. The culture has reached its maximum biomass (approx. 10 g/L) at 48 hours, remaining constant from that point forward until day 4. On day 4, glucose (40 g/L) was added to the culture, which still had about 7 g/L glucose present. MEL production was measured after 4 days (the time of addition), gradually increasing until day 14 to 8.3 g/L of MEL. This value was higher than the 5.0 g/L obtained previously also for 14 days in cultures without carbon source addition at day 4 (conditions of example 1, using glucose in place of xylose as carbon source).

### 4. Glycolipid Production From Cellulose in SHF

### 4.A. Pre-Culture

The pre-culture was prepared as described in 1.A.

### 4.B Enzyme Solution Preparation

For fermentations in SHF, it was prepared a solution of exogenous enzymes comprising mixtures commercial enzymes, Celluclast 1.5L and Novozyme 188 (Novozymes), which are described as having enzymatic activity including, but not limited to cellulase, cellobiohydrolase and betaglucosidase, respectively. The enzyme solution was prepared at a concentration three times higher than the concentration to be used in the process using 0.25% (v/v) and 1.75% (v/v) respectively. The enzyme solution was dialyzed for 24 hours at 4° C. in order to remove possible cell growth inhibitors eventually present in the commercial enzyme mixtures.

### 4.C. Enzymatic Hydrolysis of Cellulose

The SHF process was initiated with the enzymatic hydrolysis of cellulose (40 g/L) by an enzymatic solution prepared as described in section 4.B. The enzymatic hydrolysis process took place with constant mixing and at a temperature of 50° C. for 48 hours.

### 4.D. Fermentation Process for Glycolipids Production in SHF

Once the process described in 4.C. was completed, the SHF bioconversion process was carried out by adding to the solution resulting from the hydrolysis process described in 4.C. the remaining fermentation culture ingredients, with the following final concentrations:
Carbon source (cellulose added 4.D.), 40 g/L;
KH2PO₄, 0.3 g/L;
MgSO_{4.}7H2O, 0.3 g/L;
Yeast extract, 1 g/L

The medium for fermentation was prepared with sterile water at an initial pH of 6. The culture medium was then inoculated with 10% (v/v) of pre-culture prepared in 4.A. and incubated under aerobic conditions with constant mixing at 27° C. for 10 days. The process described in 4.C. lead to the release of 25.2 g/L glucose (from cellulose hydrolysis), therefore such glucose value corresponds to the fermentable sugar present in the culture at the beginning of the fermentation step, although the hydrolysis of cellulose will carry on with lower efficiency during the fermentation step. The MEL production was measured after 4 days, reaching a maximum at day 10, at a value of 4.2 g/L in MEL.

### 5. Glycolipid Production From Cellulose in SSF

### 5.A Pre-Culture

The pre-culture was prepared as described in 4.A.

### 5.B Preparation of Enzyme Solution

For fermentations in SSF, it was prepared a solution of exogenous enzymes comprising mixtures of commercial enzymes, Celluclast 1.5L and Novozyme 188 (Novozymes) as described in 4.B.

### 5.C. Fermentation Process for Glycolipids Production in SSF

Medium:
Cellulose, 40 g/l
KH2PO₄, 0.3 g/L
MgSO4.7H2O, 0.3 g/L

Yeast extract, 1 g/L

The SSF process did not include a separate enzymatic hydrolysis step at 50° C. Instead, an enzymatic hydrolysis and fermentation were initiated simultaneous with the addition of the enzyme solution prepared in S.B. and of the inoculum consisting of 10% (v/v) of the pre-culture prepared in S.A. such solution was incubated under aerobic conditions with constant mixing at 27° C. for 10 days. The MEL production was measured after 4 days, reaching a maximum at day 10, at a value of 2.94 g/L in MEL.

### 6. Glycolipid Production From Xylan/Hemicellulose Without the Addition of Exogenous Enzymes in CBP

### 6.A. Pre-Culture

The pre-culture was prepared as described in 1.A.

### 6.B Fermentation Process for Glycolipids Production in CBP

Medium:
Xylan, 40 g/L;
KH2PO₄, 0.3 g/L;
MgSO₄.7H2O, 0.3g/L;
Yeast extract, 1 g/L;

The culture medium for the fermentation step was prepared with sterile water at an initial pH of 6. The culture medium was then inoculated with 10% (v/v) of pre-culture prepared in S.A. and incubated under aerobic conditions with constant mixing at 27° C. for 10 days. The process described in the previous example covers the use of mixtures of exogenous enzymes to carry out fermentation steps with initial concentrations higher than 20 g/L (SHF) in fermentable sugar. The process described in this example does not have the addition of exogenous enzyme mixtures, exploiting the hydrolytic potential of the cells. An accumulation of 2.9 g/L of xylose after 48 hours was observed, demonstrating the cells' ability to hydrolyze xylan into simple sugars. MEL production was quantified after 4 days, reaching a maximum at day 10, at a value of 1.0 g/L of MEL.

### 7. Glycolipid Production From Wheat Straw (Agricultural Residue) in SHF

### 7.A Pre-Culture The pre-culture was prepared as described in 1.A. However, the species P. aphidis was used instead of P. antarctica.

### 7.B. Substrate Preparation

The wheat straw was submitted to a pre-treatment in a Parr reactor. A substrate to water proportion of 1:7 was used in the reactor and the reactor temperature was increased at an average rate of 6° C. per minute, with constant agitation at 150 rpm. Once the temperature reached the 210° C., the reactor was cooled down using coil to circulate cold water between the reactor and an ice bath. After 1 minute and 30 seconds, the contents of the reactor had cooled down to 100° C. Once cold, the material was filtered to recover the solid and liquid fractions. The solid fraction, rich in cellulose, was used for next bioconversion steps of the process. The total amount of glucans and xylans present in the solid fraction was 0.59 and 0.11 (g/g), respectively.

### 7.C. Preparation of Enzyme Solution

For fermentations in SHF, it was prepared a solution of exogenous enzymes comprising mixtures of the commercial enzymes Celluclast 1.5L and Novozyme 188 (Novozymes), as described in 4.B.

7.D Enzymatic hydrolysis of the solid fraction of wheat straw.

The SHF process was initiated with the enzymatic hydrolysis of solid fraction from wheat straw (7% (w/v)—dry weight per final volume of the fermentation process, as described in 6.E) prepared in 6.B. in the presence of an enzyme solution prepared as described in 6.C. The enzymatic hydrolysis step was carried out at constant mixing and 50° C. for 48 hours.

### 7.E. Fermentation Process for Glycolipids Production in SHF

Once the process described in 7.D. was completed, the SHF bioconversion process was then carried out by adding to the solution resulting from the hydrolysis process described in 6.D., the remaining fermentation culture ingredients, with the following final concentrations:
Carbon source (cellulose added in 6.D.), 40 g/L;
KH₂PO₄, 0.3 g/L;
MgSO₄.7H₂O, 0.3 g/L;
Yeast extract, 1 g/L;

The culture medium for fermentation was prepared with sterile water at an initial pH of 6.

The culture medium was then inoculated with 10% (v/v) of pre-culture prepared in 6.A. and incubated under aerobic conditions with constant mixing at 27° C. for 10 days. The process described in 6.D. lead to 24.8 g/L of glucose, which corresponds to the fermentable sugar present in the culture at the beginning of the fermentation step, although the hydrolysis of wheat straw fraction will carry on with lower efficiency during the fermentation step. The MEL production was measured after 4 days, reaching a maximum at day 10, at a value of 0.8 g/L in MEL.

### 8. Glycolipid Production From Wheat Straw (Agricultural Waste) in SSF

### 8.A. Pre-Culture

The pre-culture was prepared as described in 6.A.

### 8.B. Preparation of the Substrate

The substrate preparation was performed as described in 8.B.

### 8.C. Preparation of Enzyme Solution

For fermentations SSF was prepared a solution of exogenous enzyme comprising mixtures commercial enzymes, Celluclast 1.5L and Novozyme 188 (Novozymes), as described in 4.B.

### 8.D Process for the Fermentative Production of Glycolipids in SSF

Medium:
Fraction solid wheat straw, 7% (w/v) (dry weight per final volume)
KH₂PO₄, 0.3 g/L
MgSO₄.7H₂O, 0.3 g/L
Yeast extract, 1 g/L

The SSF process did not include a separate enzymatic hydrolysis step at 50° C. Instead, enzymatic hydrolysis and fermentation were initiated simultaneous with the addition of the enzyme solution prepared in 7.C. and of the inoculum consisting of 10% (v/v) of the pre-culture prepared in 7.A. The resulting solution was incubated under aerobic conditions with constant mixing at 27° C. for 10 days. In this SSF process was observed accumulation of 6 g/1 of glucose after 48 hours, which was consumed in the following days. The MEL production was measured after 4 days, reaching a maximum on day 10, at a value of 1.2 g/L in MEL.

### REFERENCES

1. Doble, M., & Arutchelvi, J. (2011). Biosurfactants—from genes to application. In G. S. Chávez & A. Steinbuchel (Eds.), (146-173). Springer Heidelberg Dordrecht London New York.

2. Arutchelvi J I, Bhaduri S, Uppara P V, Doble M. Mannosylerythritol lipids: a review. Journal of Industrial Microbiology & Biotechnology 35:1559-1570.

3. Kitamoto D A, Isoda H, Nakahara T (2002). Functions and potential applications of glycolipid biosurfactants—from energy-saving materials to gene delivery carriers. Journal of Bioscience and Bioengineering 94:187-201.

4. Suzuki M, Kitagawa M, Yamamoto S, et al (2010). Activator including biosurfactants as active ingredient, mannosylerythritol lipid, and production method thereof, United States Patent Application: Pub. No.: US 2010/0228013 A1.

5. Kitamoto D, Ikegami T, Suzuki G T, et al (2001). Microbial conversion of n-alkanes into glycolipid biosurfactants, mannosylerythritol lipids, by Pseudozyma (Candida) antarctica. Biotechnology Letters 23:1709-1714.

6. Rau U, Nguyen L, Roeper H, Koch H, Lang S (2005). Fedbatch bioreactor production of mannosylerythritol lipids secreted by Pseudozyma aphidis. Applied Microbiology and Biotechnology 68:607-613.

7. Morita T, Konishi M, Fukuoka T, Imura T, Kitamoto D (2007). Microbial conversion of glycerol into glycolipid biosurfactants, mannosylerythritol lipids, by a basidiomycete yeast, Pseudozyma antarctica JCM 10317T. Journal of Bioscience and Bioengineering 104:78-81.

8. Rau U, Lang S, Nguyen L, Roper H. Process for producing and recovering mannosylerythritol lipid from culture medium containing the same. International Patent Application: WO2004/020647 A1.

9. Morita T, Konishi M, Fukuoka T, Imura T, Kitamoto D (2007). Physiological differences in the formation ofthe glycolipid biosurfactants, mannosylerythritol lipids, between Pseudozyma antarctica and Pseudozyma aphidis. Applied Microbiology and Biotechnology 74:307-315.

10. Olofsson K, Bertilsson M, Lidén G (2008). A short review on SSF—an interesting process option for ethanol production from lignocellulosic feedstocks. Biotechnology for Biofuels 1:7.

11. Moldes A B, Tornado A M, Banal M T, Dominguez J M (2007) Evaluation of biosurfactant production from various agricultural residues by Lactobacillus pentosus. Journal of Agricultural and Food Chemistry 55:4481-4486.

12. Camilios-Neto D, Bugay C, de Santana-Filho A P, Joslin T, de Souza L M, Sassaki G L, Mitchell D A, Krieger N (2011) Production of rhamnolipids in solid-state cultivation using a mixture of sugar cane bagasse and corn bran supplemented with glycerol and soybean oil. Applied Microbiology and Biotechnology 89:1395-1403.

13.Cherubini F (2010) The biorefinery concept using biomass instead of oil for producing energy and chemicals. Energy Converson and Management 51:1412-1421.

1. A process for the production of microbial glycolipids, mannosylerythritol lipids, comprising:
a) using one or more carbon sources of lignocellulosic origin including cellulose, hemicellulose or mono-, di- or oligosaccharides derived from their hydrolysis, for example;
b) using one more different genera of fungi including Pseudozyma, Ustilago, Sporisorium, Moesziomyces, or Macalpinomyces as well as genetically modified fungi and bacteria including the genera Saccharomyces, Pichia, Pseudozyma, Ustilago, Escherichia and Bacillus;
c) the process uses three steps of: pretreatment, enzymatic hydrolysis and fermentation where the last two steps may take place sequentially or simultaneously, with or without addition of exogenous enzymes or their mixtures.

2. The process for the production of microbial glycolipids, mannosylerythritol lipids, according to claim 1, wherein lignocellulosic materials include wheat straw, sugar cane bagasse, sugar cane straw, corn stover, rice straw, rice husk, brewery spent grain, or paper sludge.

3. The process for the production of glycolipid, mannosylerythritol lipids, according to claim 1, wherein the preferred microorganisms for bioconversion to belong to the Pseudozyma genus.

4. The process for the production of microbial glycolipids, mannosylerythritol lipids, according to claim 3, wherein the preferred microorganisms for the bioconversion are Pseudozyma antarctica, Pseudozyma aphidis or its genetically modified variants.

5. The Process for the production of microbial glycolipids, of mannosylerythritol lipids, according to claim 1, wherein the lignocellulosic material is submitted to a physical and/or chemical treatment which comprises the use of acids, bases, organic solvents and/or water and the combination of different temperatures and reaction times, between 100 and 250° C. and 0 to 300 minutes, respectively.

6. The process for the production of microbial glycolipids, mannosylerythritol lipids, according to claim 1, wherein the lignocellulosic material is treated with exogenous enzymes or their mixtures with activities including cellulase, cellobiohydrolase hydrolase glucosidase, xylanase, xylosidase and arabinofuranosidase.

7. The process according to claim 6, wherein the exogenous enzymes or their mixtures are submitted to a purification step.

8. The process for the production of microbial glycolipids, mannosylerythritol lipids, according to claim 1, wherein the enzymatic hydrolysis and the fermentation step take place sequentially in different reaction vessels or in the same reaction vessel, with the enzymatic hydrolysis temperature between 20 and 80° C. and respective pH between 3 and 7, and the fermentation temperature between 4 and 40° C. and respective pH between 3 and 7.

9. The process for the production of microbial glycolipids, mannosylerythritol lipids, according to claim 1, wherein enzymatic hydrolysis and fermentation result take place sequentially in different reaction vessels or in the same reaction vessel, with the enzymatic hydrolysis step taking place preferably at a temperature of 50° C. and pH 5.5, and fermentation step taking place preferably at a temperature of 27° C. and pH 5.5.

10. The process for the production of microbial glycolipids, mannosylerythritol lipids, according to claim 1, wherein the enzymatic hydrolysis and fermentation process take place simultaneously in the same reaction vessel, at a temperature between 4 and 40° C. and pH between 3 and 7.

11. The process for the production of microbial glycolipids, mannosylerythritol lipids, according to claim 1, wherein the enzymatic hydrolysis and fermentation process take place simultaneously in the same reaction vessel, preferably at a temperature of 27° C. and pH 5.5.

12. The process for the production of microbial glycolipids, mannosylerythritol lipids, according to claim 1, wherein the enzymatic hydrolysis occurs by action of endogenous enzymes produced by the microorganism used in the fermentation process.

13. The process for the production of microbial glycolipids, mannosylerythritol lipids, according to claim 1, wherein the enzymatic hydrolysis occurs through the combined use of the endogenous enzymes, produced by the microorganism used in the fermentation process, with addition of exogenous enzymes or their mixtures.

14. The process for the production of microbial glycolipids according to claim 1 wherein the carbon sources of lignocellulosic origin are selected from the group consisting of mannosylerythritol lipids celodextrinas, xylooligosaccharide, cellobiose, xylobiose, D-glucose, D-mannose, D-galactose, D-xylose and L-arabinose
